(19) United States
(12) Patent Application Publication
(10) Pub. No.: US 2021/0177746 A1
(43) Pub. Date: Jun. 17, 2021

(54) OPHTHALMIC SOLUTION OF ANTIOXIDANT

(71) Applicant: Tathagata DUTTA, BERHAMPORE (IN)

(72) Inventor: Tathagata DUTTA, BERHAMPORE (IN)

(21) Appl. No.: 16/636,305

(22) PCT Filed: Mar. 26, 2018
(86) PCT No.: PCT/IN2018/050171

§ 371 Date: Feb. 3, 2020

(30) Foreign Application Priority Data
Aug. 10, 2017 (IN) 201741028405

(51) Int. Cl. A61K 9/00; A61K 31/047; A61K 47/26; A61K 47/10; A61K 47/18; A61K 47/02; A61K 47/36; A61K 47/34; A61K 47/12; A61K 31/355; A61K 33/04; A61K 47/20; A61K 9/08

(52) U.S. Cl. CPC A61K 9/0048; A61K 31/047; A61K 47/26; A61K 47/10; A61K 47/186; A61K 47/02; A61K 9/08; A61K 47/34; A61K 47/12; A61K 31/355; A61K 33/04; A61K 47/20; A61K 47/36

(57) ABSTRACT

A topical ocular solution for treating or preventing age related macular degeneration (ARMD) comprising of one or more antioxidants and at least one pharmaceutically acceptable excipient, wherein the antioxidant is a carotenoid selected from lutein, zeaxanthin or antioxidants like alpha tocopherol and antioxidants of mineral origin like selenium or a combination thereof. An ophthalmic solution of antioxidant for treating or preventing age related macular degeneration (ARMD) comprising of one or more antioxidants, a surfactant/solubilizing agent, additionally at least one pharmaceutically acceptable excipient selected from the group comprising of a pH adjusting agent, a buffering agent, a osmolarity control agent and a preservative, wherein the ophthalmic solution has a pH range of 4 to 8.

### FIELD OF THE INVENTION

This invention relates to drug delivery dosage forms and methods to treat medical conditions of the eye. Specifically, this invention relates to solution drug delivery dosage forms of antioxidants for drug delivery within the eye.

### BACKGROUND OF THE INVENTION

Age related macular degeneration (ARMD) is a severe problem of the eye which accounts for the loss of vision of huge number of elderly population across the globe. The disease which starts as a mild innocent problem of the eye, including occasional floaters and black dots in front of the eye gradually progresses to the loss of peripheral vision to complete loss of vision.

Antioxidants are well known to slow down the progression of the ARMD disease. There are several formulations available in the market containing single or combination of antioxidants like Alpha Tocopherol, Lutein, Zeaxanthin, Selenium, etc for oral administration for slowing down the progression of the disease. There are several drawbacks with orally administered drugs or bioactives as the drug does not reach the eye at appropriate concentrations and has either none or very poor pharmacological action in the eye when administered through oral route.

Hence, there is a need for a topical formulation of antioxidants like Lutein and/or Zeaxanthin for ocular administration that will release the drug at immediately |thereby|_{[TD1][TD2]} maintaining a steady concentration of antioxidants in the aqueous and vitreous humor thereby increasing the macular pigment optical density (MPOD), which will be helpful for better management of ARMD.

### SUMMARY OF THE INVENTION

Accordingly, the invention provides an ophthalmic solution of antioxidant for treating or preventing age related macular degeneration (ARMD) comprising of one or more antioxidants, a surfactant/solubilizing agent, additionally at least one pharmaceutically acceptable excipient selected from the group comprising of a pH adjusting agent, a buffering agent, a osmolarity control agent and a preservative, wherein the ophthalmic solution has a pH range between 4 to 8. The ophthalmic solution as described herein is an emulsion or solution.

The ophthalmic solution of antioxidant for treating or preventing age related macular degeneration (ARMD) described herein comprises the antioxidant in a concentration of about 0.003% to 6.0% w/v. The antioxidant is lutein, zeaxanthin, alpha tocopherol, selenium or a combination thereof. The surfactant/solubilising agent is a Polyoxyethylated nonionic surfactant and include Polysorbate80, Polysorbate 60, Polysorbate, 40, Polysorbate 20, Cremophors, Tyloxapols, Poloxamers, Benzalkonium chloride, Benzethonium chloride, Cetyl alcohol, carbomer, cholesterol, Cocamidopropyl betaine, glyceryl monostearate, lanolin alcohols, lauralkonium chlorides, N lauroylsarcosine, Nonoxynol 9, Octoxynol 40, Polyoxyl 35 castor oil, Polyoxyl 40 hydrogenated castor oil, Polyoxyl 40 stearate, Sorbitanmonolaureate, Sorbitan monooleate or a combination thereof. The pH adjusting agent is selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof. The osmolarity control agent is selected from the group comprising of Sodium Chloride, Sodium Sulphate, Sodium Nitrate, Sorbitol, Mannitol, Calcium Chloride, Glycerine, Magnesium chloride, PEG 300, PEG 400, Potassium Chloride, Propylene Glycol or a combination thereof. The buffering agent is selected from the group comprising of Acetic Acid, Boric Acid, Citric Acid, Phosphoric Acid, Potassium Acetate, Potassium Phosphate, Potassium Sulphate, Potassium Sorbate, Sodium Acetate, Sodium borate, Sodium Carbomate, Sodium Citrate, Sodium Phosphate, Sorbic Acid, Tromethamine or a combination thereof. The preservative is selected from the group comprising of Quaternary ammonium compounds, Acid/Base compounds, Alcohols, Organic Mercuric compounds, Parabens, Oxidizing agents and Metal salts.

### DETAILED DESCRIPTION OF THE INVENTION

The ophthalmic solution of antioxidant for treating or preventing age related macular degeneration (ARMD) as described herein comprises pharmaceutically effective amount of one or more antioxidants. The antioxidant is lutein, zeaxanthin, alpha tocopherol, selenium or a combination thereof.

The ophthalmic solution as described herein comprises the antioxidant in a concentration of about 0.003% to 6.0% w/v and the ophthalmic solution has a pH range of 4 to 8.

The Ophthalmic solution according to the invention described herein contains surfactants/solubilizing agents. Many ophthalmically and otically acceptable polyoxythylated nonionic surfactants are known. These non-ionic surfactants include Polysorbate 80, Polysorbate 60, Polysorbate, 40, Polysorbate 20, Cremophors, Tyloxapols, Poloxamers, Benzalkonium chloride, Benzethonium chloride, Cetyl alcohol, carbomer, cholesterol, Cocamidopropyl betaine, glyceryl monostearate, lanolin alcohols, lauralkonium chlorides, N lauroylsarcosine, Nonoxynol 9, Octoxynol 40, Polyoxyl 35 castor oil, Polyoxyl 40 hydrogenated castor oil, Polyoxyl 40 stearate, Sorbitan monolaureate, Sorbitan monooleate or a combination thereof.

The ophthalmic solution of antioxidant as described herein also comprises at least one pharmaceutically acceptable excipient selected from the group comprising of a pH adjusting agent, a buffering agent, a osmolarity control agent and a preservative.

The ophthalmic solution according to the invention described contains a pH adjusting agent. The pH adjusting agent is selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof.

The ophthalmic solution according to the invention described contains an osmolarity control agent. The Osmolarity control agent is selected from the group comprising of Sodium Chloride, Sodium Sulphate, Sodium Nitrate, Sorbitol, Mannitol, Calcium Chloride, Glycerine, Magnesium chloride, PEG 300, PEG 400, Potassium Chloride, Propylene Glycol or a combination thereof.

The ophthalmic solution according to the invention described contains a buffering agent. The buffering agent is selected from the group comprising of Acetic Acid, Boric Acid, Citric Acid, Phosphoric Acid, Potassium Acetate, Potassium Phosphate, Potassium Sulphate, Potassium Sorbate, Sodium Acetate, Sodium borate, Sodium Carbomate, Sodium Citrate, Sodium Phosphate, Sorbic Acid, Tromethamine or a combination thereof.

The ophthalmic solution according to the invention described also contains a preservative. The preservative is selected from the group comprising of Quaternary ammonium compounds, Acid/Base compounds, Alcohols, Organic Mercuric compounds, Parabens, Oxidizing agents and Metal salts. The Quaternary ammonium compounds include Benzalkonium Chloride, Benzethonium chloride, Benzododecinium bromide, Polyquatermium-1, etc. The Acid/Base compounds include Boric acid, sodium acetate, sodium borate, etc. The Alcohols include chlorobutanol, Phenylethylalcohol, etc. The Organic Mercuric compounds include Phenyl mercuric acetate, Phenyl mercuric nitrate, Thimerosal, etc, Parabens include methyl paraben, Propyl Paraben, etc.

In one embodiment, the ophthalmic solution comprises lutein and zeaxanthin.

### Example: 1

### Lutein+Zeaxanthin Ophthalmic Solution

| S. No | Ingredient | mg/mL | |
|---|---|---|---|
| 1 | Lutein | 0.003-3 | mg |
| 2 | Zeaxanthin | 0.003-3 | mg |
| 3 | Polysorbate 80 | 4 | mg |
| 4 | Propylene Glycol | 50 | mg |
| 5 | Polyethylene Glycol 400 | 50 | mg |
| 6 | Benzalkonium Chloride | 0.001% | |
| 7 | Water for Injection | q.s | |
| 8 | Sodium Chloride | 0.6 | mg |
| 9 | Hydrochloric acid/sodium | q.s | |
| | hydroxide | | |

Process: Required quantities of lutein and zeaxanthin are mixed with polysorbate 80, propylene glycol, polyethylene glycol 400 and benzalkonium chloride. To this mixture was added sodium chloride and the volume was adjusted by water for injection. The pH was adjusted between 4 to 8 by addition of hydrochloric acid or sodium hydroxide.

In another embodiment, the ophthalmic solution comprises lutein.

### Example: 2

### Lutein Ophthalmic Solution

| S. No | Ingredient | mg/mL | |
|---|---|---|---|
| 1 | Lutein | 0.003-3 | mg |
| 2 | Poloxamer 407 | 2 | mg |

| | | |
|---|---|---|
| 3. | Disodium Edetate | 0.13% |
| 4. | Phenyl Mercuric Nitrate | 0.002% |
| 5. | Sodium Chloride | 0.03% |
| 6. | Hydrochloric Acid/Sodium Hydroxide | q.s |
| 7. | Water for Injection | q.s |

Process: Required quantity of Lutein is mixed with Poloxamer 407 and Disodium EDTA. To the mixture so obtained, Phenyl Mercuric Nitrate was added as a preservative and sodium chloride as Osmolarity adjusting agent and mixed with water for injection. The solution so formed is made up to the volume with water for injection & the pH was adjusted using hydrochloric acid/sodium hydroxide and aseptically filled into containers, and sealed.

In another embodiment of the invention, the ophthalmic solution comprises zeaxanthin.

### Example: 3

### Zeaxanthin Ophthalmic Solution

| S. No | Ingredient | mg/mL | |
|---|---|---|---|
| 1 | Zeaxanthin | 0.003-3 | mg |
| 2 | Tyloxapol | 3 | mg |
| 3 | Sodium citrate | 0.5-5 | mg |
| 4 | Citric Acid | 0.3-1.5 | mg |

| | | |
|---|---|---|
| 5 | Benzalkonium chloride | 0.001% |
| 6 | Absolute alcohol | 1.2% w/v |
| 7 | Water for injection | q.s |
| 8 | Sodium hydroxide/hydrochloric | q.s |
| | acid | |

Process: Required quantity of Zeaxanthin is mixed with Tyloxapol, absolute alcohol, sodium citrate & citric acid. To the mixture so obtained, Benzalkonium chloride was added as a preservative and mixed with water for injection. The solution so formed is made up to the volume with water for injection & the pH was adjusted using hydrochloric acid/sodium hydroxide and aseptically filled into containers, and sealed.

In another embodiment of the invention, the ophthalmic solution comprises lutein, zeaxanthin, alpha tocopherol and selenium.

### Example: 4

### Lutein, Zeaxanthin, Alpha Tocopherol & Selenium Ophthalmic Solution/Drops

| S. No | Ingredient | mg/mL | |
|---|---|---|---|
| 1 | Lutein | 0.003-3 | mg |
| 2 | Zeaxanthin | 0.03-6 | mg |
| 3 | Alpha Tocopherol | 0.005-0.5 | mg |
| 4 | Selenium | 0.001-0.01 | mg |
| 5 | Poloxamer 188 | 1 | mg |
| 6 | Polysorbate 80 | 40 | mg |
| 7 | Polyethylene Glycol (PEG) 8000 | 20 | mg |
| 8 | Polyethylene Glycol (PEG) 400 | 50 | mg |
| 9 | Sodium chloride | 0.5 | mg |

| | | |
|---|---|---|
| 10 | Phenyl mercuric nitrate | 0.002% |
| 11 | Water for Injection | q.s |
| 12 | Sodium Hydroxide/Hydrochloric Acid | q.s |

Process: Required quantities of Lutein, Zeaxanthin, Alpha Tocopherol & Selenium were well mixed with PEG 8000& Polysorbate 80, followed by addition of Poloxamer 188, Phenyl mercuric nitrate & sodium chloride. To the mixture so obtained, was added required quantity of PEG 400, followed by addition of required quantity of water for Injection along with continuous stirring. The pH was adjusted with sodium hydroxide/hydrochloric acid and filtered aseptically, and filled and sealed.

In another embodiment of the invention, the ophthalmic solution comprises lutein and zeaxanthin.

### Example: 5

### Lutein+Zeaxanthin Ophthalmic Solution/Drops

| S. No | Ingredient | mg/mL | |
|---|---|---|---|
| 1 | Lutein | 0.003-3 | mg |
| 2 | Zeaxanthin | 0.003-3 | mg |
| 3 | Poloxamer 407 | 2 | mg |
| 4 | Absolute Alcohol | 1.4% w/v | |
| 5 | Sodium Sulphate | 0.9 | mg |
| 6 | Sodium Citrate | 0.2 | mg |
| 7 | Citric Acid | 0.6 | mg |

| | | |
|---|---|---|
| 8 | Benzalkonium Chloride | 0.001% |
| 9 | Hydrochloric Acid/Sodium | q.s |
| | Hydroxide | |
| 10 | Water for Injection | q.s |

Process: Required quantities of Lutein & Zeaxanthin were well mixed with Poloxamer 407&absolute alcohol, followed by addition of sodium citrate, citric acid & sodium sulphate. To the mixture so obtained, was added required quantity of Benzalkonium chloride, followed by mixing with required quantity of water for Injection along with continuous stirring. The pH was adjusted using required quantities of sodium hydroxide/hydrochloric acid. The solution so formed is aseptically filtered, followed by filling and sealing.

The method for preparing the ophthalmic solution of antioxidants according to the disclosure described herein is not limited to the above methods. The ophthalmic solutions of antioxidants according to the disclosure described herein can be prepared by using various other techniques.

We claim:

1. An ophthalmic solution of antioxidant for treating or preventing age related macular degeneration (ARMD) comprising of one or more antioxidants, a surfactant/solubilizing agent, additionally at least one pharmaceutically acceptable excipient selected from the group comprising of a pH adjusting agent, a buffering agent, a osmolarity control agent and a preservative, wherein the ophthalmic solution has a pH range of 4 to 8.

2. The ophthalmic solution according to claim 1, wherein the concentration of antioxidant is about 0.003% to 6.0% w/v.

3. The ophthalmic solution according to claim 1, wherein the antioxidant is selected from lutein, zeaxanthin, alpha tocopherol, selenium or a combination thereof.

4. The ophthalmic solution according to claim 1, wherein the surfactant/solubilising agent is selected from the group comprising of Polysorbate80, Polysorbate 60, Polysorbate, 40, Polysorbate 20, Cremophors, Tyloxapols, Poloxamers, Benzalkonium chloride, Benzethonium chloride, Cetyl alcohol, carbomer, cholesterol, Cocamidopropyl betaine, glyceryl monostearate, lanolin alcohols, lauralkonium chlorides, N lauroylsarcosine, Nonoxynol 9, Octoxynol 40, Polyoxyl 35 castor oil, Polyoxyl 40 hydrogenated castor oil, Polyoxyl 40 stearate, Sorbitanmonolaureate, Sorbitan monooleate or a combination thereof.

5. The ophthalmic solution according to claim 1, wherein the pH adjusting agent is selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof.

6. The ophthalmic solution according to claim 1, wherein the osmolarity control agent is selected from the group comprising of Sodium Chloride, Sodium Sulphate, Sodium Nitrate, Sorbitol, Mannitol, Calcium Chloride, Glycerine, Magnesium chloride, PEG 300, PEG 400, Potassium Chloride, Propylene Glycol or a combination thereof.

7. The ophthalmic solution according to claim 1, wherein the buffering agent is selected from the group comprising of Acetic Acid, Boric Acid, Citric Acid, Phosphoric Acid, Potassium Acetate, Potassium Phosphate, Potassium Sulphate, Potassium Sorbate, Sodium Acetate, Sodium borate, Sodium Carbomate, Sodium Citrate, Sodium Phosphate, Sorbic Acid, Tromethamine or a combination thereof.

8. The ophthalmic solution according to claim 1, wherein the preservative is selected from the group comprising of Quaternary ammonium compounds, Acid/Base compounds, Alcohols, Organic Mercuric compounds, Parabens, Oxidizing agents and Metal salts.

9. The ophthalmic solution according to claim 1, wherein the ophthalmic solution is a suspension or solution.
